(19) United States
(12) Patent Application Publication
(10) Pub. No.: US 2004/0241141 A1
(43) Pub. Date: Dec. 2, 2004

(54) Method of treating arthritis using lentiviral vectors in gene therapy

(75) Inventors: Robert Pawliuk, Medford, MA (US); Philippe Leboulch, Charlestown, MA (US)

Correspondence Address: LAHIVE & COCKFIELD, LLP., 28 STATE STREET, BOSTON, MA, 02109, US

(73) Assignee: Genetix Pharmaceuticals, Inc., Cambridge, MA (US)

(21) Appl. No.: 10/688,780

(22) Filed: Oct. 15, 2003

Related U.S. Application Data
(63) Continuation of application No. PCT/US02/08711, filed on Mar. 21, 2002.
(60) Provisional application No. 60/284,736, filed on Apr. 17, 2001.

(51) Int. Cl. A61K 48/00

(52) U.S. Cl. 424/93.2

(57) ABSTRACT

Novel methods for treating and preventing arthritis, such as rheumatoid arthritis, are disclosed which employ lentiviral gene delivery vectors, including HIV-based lentiviral vectors, to deliver a therapeutic gene to a subject. Lentiviral-based vectors treat arthritis by promoting high-level expression of the transferred therapeutic gene in the target tissue of the subject.

### RELATED APPLICATIONS

This application is a continuation of PCT/US02/08711 filed Mar. 21, 2002 which claims the benefit of PCT/US02/08600, filed Mar. 19, 2002 and U.S. provisional application No. 60/284,736 filed Apr. 17, 2001 all of which are hereby incorporated by reference.

### BACKGROUND OF THE INVENTION

Arthritis (both osteoarthritis [OA] and rheumatoid arthritis [RA]), the most prevalent musculoskeletal disorder (Martel-Pelletier et al. (1999) Frontiers in Bioscience 4:d694-703), is characterized by the progressive destruction of articular cartilage and concurrent proliferation of bone, cartilage and connective tissue cells. This progressive destruction and proliferative response leads to the destabilization and remodeling of the entire joint structure resulting in pain, inflammation, stiffness and a restriction in movement (Martel-Pelletier et al. (1999), supra). By the age of 65 approximately 80% of people show some radiographic evidence of OA (Nuki et al (1999) Davidson's Principle and Practice of Medicine p. 826).

Current therapy for OA and RA includes the use of analgesics, such as non-steroidal anti-inflammatory drugs, or intra-articular injections of hyaluronan or corticosteroids for temporary relief of pain and inflammation. Such treatments, however, can be associated with numerous side-effects including gastric erosion or hemorrhage, impairment of renal function, osteoporosis and hypertension (Nuki et al., supra). In patients with advanced OA surgical intervention is required to provide relief from pain and disability. All of the aforementioned therapies however, are aimed at treating the symptoms of the disease and are not curative.

Over the past decade significant progress has been made in the identification of molecules which play a key role in the initiation/progression of OA and RA (Martel-Pelletier et al. (1999), supra). Although the initiating event in OA/RA remains controversial, it is now clear that the destruction of articular cartilage occurs as the result of an imbalance between catabolic (destructive) and anabolic (productive) factors (Malemud and Goldberg, (1999) Frontiers in Bioscience 4:d762-771). Examples of catabolic factors include Interleukin (IL)-1 beta, IL-6, Leukemia Inhibitory factor (LIF), Tumor Necrosis Factor (TNF)-alpha, fibronectin fragments, urokinase plasminogen activator and Matrix Metallo-Proteinases (MMPs). Anabolic factors include Transforming Growth Factor (TGF)-beta, Insulin Growth Factor (IGF)-1, Platelet Derived Growth Factor (PDGF), IL-4, IL-10, IL-11, IL-13, Bone Morphogenic Protein (BMP)-2, BMP-7 and Tissue Inhibitors of Matrix Metallo-Proteinases (TIMPs) (Martel-Pelletier et al. (1999), supra; Malemud and Goldberg, supra). The identification of molecules critical to the progression of OA has led to efforts aimed at preventing or even reversing the destruction of articular cartilage.

To date, several groups have investigated the efficacy of inhibiting the effects of catabolic cytokines using protein antagonists of cell surface receptors, soluble receptors or antibodies against cytokines or their receptors in pre-clinical models (Bessis et al., (2000) Eur. J. Immunol. 30:867; Caron et al., (1996) Arthritis Rheum. 39:1535) and clinical trials (Bresnihan et al. (1998) Arthritis Rheum. 41:2196; McKay et al. (1998) Arthritis Rheum. 41:S132; Elliot et al. (1994) Lancet 344:1105; Moreland et al. (1999) Ann. Inter. Med. 16: 478; Moreland et al. (1997) New Eng. J. Med. 337:141). A serious limitation to this approach, however, is the short half-life and efficacy of the administered proteins. For example, although arthritic patients showed significant and rapid improvement upon treatment with soluble TNF-alpha receptor, all benefits were quickly reversed upon withdrawal of treatment (Moreland et al. (1997), supra). Moreover, these proteins can be difficult to administer and must be administered frequently. This observation illustrates the requirement for high-level, long-term, stable production of the therapeutic protein within the affected joint.

Gene therapy is currently being investigated as an alternative approach to the treatment of arthritis. Indeed, several studies in animals have provided experimental evidence both ex vivo and in vivo demonstrating the feasibility and/or efficacy of gene therapy using recombinant adenovirus (rAAV) (Lubberts et al. (1999) J. Immunol. 163:4546; Taniguchi et al. (1999) Nat. Med. 5:760; Ikeda et al. (1998) J. Rheumatol. 25:1666; Zhang et al. (1997) J. Clin. Invest. 100:1951; Whalen et al. (1999) J. Immunol. 162:3625; Baragi et al. (1995) J. Clin. Invest. 96: 2454; Kobayashi et al. (2000) Gene Ther. 7:527; Smith et al. (2000) Arthritis Rheum. 43:1156; Ghivizzani et al. (1998) Proc. Natl. Acad. Sci. USA 95:4613), adeno-associated virus (AAV) (Arai et al. (2000) J. Rheumatol. 27:979; Goater et al. (2000) J. Rheumatol. 27:983), retrovirus (Muller-Ladner et al. (1997) J. Immunol. 158:3492; Makarov et al. (1996) Proc. Natl. Acad. Sci. USA 93:402), Moloney monkey leukemia virus (MoMLV) (Ghivizzani et al. (1997) Gene Ther. 4:977-982; Nguyen et al. (1998) J. Rheumatol. 25:1118-1125), or naked DNA (Sant et al. (1998) Hum. Gene Ther., 9:2735; Fernandes et al. (1999) Am. J. Path. 54:1159; Song et al. (1998) Clin. Invest. 101:2615), and several clinical trials for gene therapy of rheumatoid arthritis have been initiated.

Although various strategies have been tested, those that target gene delivery to the synovial lining of the joints (Bandara et al. (1992) DNA Cell Biol., 11:227-231; Bandara et al. (1993) Proc. Natl. Acad. Sci. USA, 90:10764-10768) have made the most experimental progress. This strategy has shown efficacy in several models of RA (Ghivizzani et al. (1998), supra; Kim et al. (2000) Arthritis Res. 2:293-302; Makarov et al., supra; Whalen et al., supra; Yao et al. (2001) Mol. Ther. 3:901-903; Otani et al. (1996) J. Immunol. 156:3558-3562; Hung et al. (1994) Gene Ther. 1:64-69). Moreover, in two clinical studies it has proved possible to transfer safely the human IL-1Ra cDNA to human rheumatoid joints (Evans et al. (1996) Hum. Gene Ther. 7:1261-1280; Evans et al. (2000) Clin. Orthop. S300-307). These protocols utilized an ex vivo approach involving transduction of autologous synovial fibroblasts with a vector derived from the MoMLV. While useful for establishing proof of concept, ex vivo methods are labor intensive and expensive, and thus do not lend themselves well to widespread clinical application. For this reason, increasing attention has been brought to developing clinically acceptable in vivo methods of gene delivery to synovium.

In preclinical experiments several vectors, either viral or non-viral, have been used to transfer exogenous genes to synovium by in vivo delivery (Ghivizzani et al. (2001) Drug Discov. Today 6:259-267). Among them, two appear particularly promising; rAAV and high-titer MoMLV (Ghivizzani et al. (1997), supra; Nguyen et al., supra). RAAV encodes no viral proteins, is not inflammatory, and is able to infect both dividing and non-dividing cells. In some cells, but not all, rAAV has been found to integrate the genome of the target cells (Hirata et al. (2000) J. Virol. 74:4612-4620) and provide long term transgene expression. However, despite recent technological progress, high-titer rAAV vectors are difficult to generate (Monahan et al. (2000) Mol. Med. Today 6:433-440), a limitation that has hindered their evaluation as a vector for gene delivery to joints. Moreover, the literature reports widely divergent results from experiments attempting in vivo gene delivery to joints with AAV-based vectors (Ghivizzani et al. (2001), supra). MoMLV-based oncoretroviruses efficiently and permanently integrate into the genome of transduced target cells and are therefore particularly attractive for chronic conditions such as RA that will probably require extended periods of intra-articular expression. However, they require mitosis of the target cell for successful transduction (Lewis et al. (1994) J. Virol. 68:510-516), limiting their efficient in vivo delivery to conditions, such as acute inflammation, where many cells within synovium are rapidly dividing (Ghivizzani et al. (1997), supra; Nguyen et al., supra).

Due to the inefficient and/or non-integrative properties of naked DNA, rAAV, and adenoviruses, as well as the difficulty in generating high-titer rAAV vectors, these vectors are unable to provide long term expression of the therapeutic proteins in vivo. In addition, due to their inability to efficiently transduce non-dividing cells such as synovial fibroblasts and chondrocytes, MoMLV-based oncoretrovirus vectors are not the best candidates for providing long term therapy of arthritis. Most importantly, none of the existing gene delivery systems have been able to achieve long-term expression of the transgene intra-articularly.

In contrast to oncoretroviruses, lentiviruses, including the human immunodeficiency virus (HIV), feline immunodeficiency virus (FIV), and simian immunodeficiency virus (SIV), are able to efficiently infect and stably transduce cells that have terminally differentiated and/or are non-dividing (Lewis, et al. (1994), supra; Lewis et al. (1992) EMBO J. 11:3053-3058; Naldini et al. (1996) Science 272:263-267; Bukrinsky et al. (1993) Nature 365:666-669). Although the use of HIV-based viruses for in vivo gene therapy seems encouraging, the complexity of their biology and safety concerns have complicated and slowed their clinical application (Buchschacher et al. (2000) Blood 95:2499-2504; Naldini et al. (1998) Curr. Opin. Biotechnol. 9:457-463; Vigna et al. (2000) J. Gene Med. 2:308-316). To reduce potential risks, multiply attenuated systems have been developed where up to six viral genes, those essential for HIV replication and pathogenesis, have been inactivated or deleted (Zufferey et al. (1997) Nat. Biotechnol. 15:871-875; Kim et al. (1998) J. Virol. 72:811-816; Gasmi et al. (1999) J. Virol. 73:1828-1834). Using a third generation packaging system, it is now possible to produce high-titer (>10⁹ iu/ml) replication incompetent, HIV-based retroviruses with a high level of expected biosafety, which may be acceptable for clinical application (Vigna et al., supra; Dull et al. (1998) J. Virol. 72:8463-8471). The latest generation of lentiviral vectors has also been shown to transduce with high efficiency CD34+ hematopoietic stem cells (Akkina et al. (1996) J. Virol. 70:2581-2585; Case et al. (1999) Proc. Natl. Acad. Sci. USA 96:2988-2993). Advances in the use of lentivirus-based vectors, like HIV, in gene therapy provide additional methods for preventing and treating arthritis.

### SUMMARY OF THE INVENTION

The present invention provides an improved method for treating arthritis using a lentiviral gene delivery system which exhibits sustained, high-level expression of transferred therapeutic genes in vivo. Lentiviral vectors employed in the gene delivery system of the present invention are highly efficient at infecting and integrating in a non-toxic manner into the genome of a wide variety of cell types, including chondrocytes and synovial fibroblasts.

Suitable lentiviral vectors for use in the invention include, but are not limited to human immunodeficiency virus (HIV-1, HIV-2), feline immunodeficiency virus (FIV), simian immunodeficiency virus (SIV), bovine immunodeficiency virus (BIV), and equine infectious anemia virus (EIAV). In one embodiment, the vector is made safer by separating the necessary lentiviral genes (e.g., gag and pol) onto separate vectors as described, for example, in U.S. patent application Ser. No. 09/311,684, the contents of which are incorporated by reference herein. In another embodiment, the vector is made safer by replacing certain lentiviral sequences with non-lentiviral sequences. Thus, lentiviral vectors of the present invention may contain partial (e.g., split) gene lentiviral sequences and/or non-lentiviral sequences (e.g., sequences from other retroviruses) as long as its function (e.g., viral titer, infectivity, integration and ability to confer sufficient levels and duration of therapeutic gene expression) are not substantially reduced.

In order to increase their target cell range and to facilitate concentration by centrifugation, the lentiviral vectors of the invention can be pseudotyped with an envelope protein, such as the vesicular stomatitis virus G-protein (VSV-G), using known techniques in the art (see e.g., Chesebro et al. (1990) J. Virol. 64 (1): 215-221; Naldini et al. (1996), supra; U.S. Pat. No. 5,665,577 (Sodroski et al.); and WO 97/17457 (Salk Institute). The lentiviral gene delivery system of the present invention also can be used in conjunction with a suitable packaging system able to produce high titers of replication-incompetent lentiviral-based retroviruses.

In a particular embodiment of the invention, the lentiviral vector contains a therapeutic gene which can be expressed in the target tissue at sufficient levels and for a sufficient level of time to prevent or reverse the destruction of articular cartilage, as occurs in arthritis. In a further embodiment of the invention, the lentiviral vector is selected from a group consisting of HIV, FIV, SIV, BIV, and EIAV vectors. Examples of suitable therapeutic genes which can be delivered in vivo to treat arthritis in accordance with the present invention include, but are not limited to, the following: soluble interleukin-1 receptors, antagonists of the interleukin-1 receptors, soluble TNF-α receptors, fibronectin and fibronectin fragments, TGF-β family members, IGF-1, LIF, BMP-2, BMP-7, plasminogen activators, plasminogen inhibitors, MMPs, TIMPs, Indian Hedgehog, parathyroid hormone-related protein, IL-4, IL-10, IL-11, IL-13, hyaluronan synthase, and PDGF-BB. Accordingly, the lentiviral vectors can be delivered in vivo to a subject having arthritis (e.g., rheumatoid arthritis (RA)). In one embodiment, the vectors are delivered into the synovial lining of affected joints by, for example, direct injection (e.g., intra-articular). This provides extended (e.g. intra-articular) gene integration and expression.

In another embodiment, the lentiviral vectors can be used to treat arthritis by transfecting either autologous or non-autologous, including allogeneic or xenogeneic, cells ex vivo which can then be delivered to a subject (e.g., injected into arthritic joints or other affected areas). Suitable autologous cells include, for example, bone marrow cells, mesenchymal stem cells obtained from adipose tissue, and synovial fibroblasts or chondrocytes. Suitable non-autologous cells include, for example, cell lines and primary cells derived from a human or animal source.

### BRIEF DESCRIPTION OF THE FIGURES

FIG. 1 is a schematic representation of the β-GEO (A) and hIL-1Ra (B) lentiviral vectors. HIV LTR, human immunodeficiency virus long terminal repeat; Ψ+, packaging signal; RRE, Rev-responsive element; cPPT/FLAP, central polypurine tract/DNA flap; PPT, polypurine tract. Expression of the gene of interest is under the control of the EF-1α promoter.

FIG. 2 shows lentivirus-mediated delivery of the hIL-1Ra gene in vitro and in vivo. Panel (A) is a graph showing in vitro expression levels of hIL-1Ra following infection of 10⁵ rat synovial cells using a range of multiplicities of infection (MOI) of hIL-1Ra lentivirus. Panel (B) is a graph showing in vivo expression levels of hIL-1Ra after intra-articular injection of lentivirus into the knee joint of immuno-compromised rats (solid bars) or normal Wistar rats (clear bars). Each bar represents mean values±S.D. from 8 knees of 4 rats. (*P<0.01 compared to hIL-1Ra levels in Wistar rats, t-test). Panel (C) is a graph showing in vivo expression levels of hIL-1Ra after intra-articular injection of recombinant lentivirus into the knee joint of immuno-compromised (nude) rats.

FIG. 3 is a graph showing the biodistribution of the hIL-1Ra protein following the intra-articular injection of 5×10⁷ iu IL-1Ra lentivirus. Naïve animals (clear bars) were compared to rats sacrificed 5 (gray bars) and 10 (black bars) days post-injection. Each bar represents mean values±S.D. from 6 rats. (*P<0.01, t-test).

FIG. 4 shows graphs of local (knee diameter) and systemic (body weight) effects of lentivirus-mediated hIL-1Ra expression on arthritic rats injected with 3×10³ (A), 10⁴ (B), 3×10⁴ (C) or 105 (D) dermal fibroblasts engineered to produce hIL-1β. White bars, normal knees; Black bars, arthritic knees; Grey bars, lentivirus-injected arthritic knees; Striped bars, contralateral arthritic knees. (Insets) Evolution of rat body weight overtime. White diamonds, naive rat; Grey triangles, lentivirus-treated arthritic rat; Black squares, arthritic rat. The results were expressed as the mean±SD from 8-11 rats. (*P<0.01 compared to arthritic rats, t-test).

### DETAILED DESCRIPTION OF THE INVENTION

The present invention provides improved compositions and methods for gene therapy, particularly in the treatment of arthritis. As described in detail below, lentiviral vectors are used to deliver therapeutic genes to affected cells or tissues, thereby providing sustained, high level expression of therapeutic proteins to selected areas of treatment.

In order that the present invention may be more readily understood, certain terms are first defined. Additional definitions are set forth throughout the detailed description.

I. Definitions

As used herein, the term “arthritis” includes any disease characterized by inflammation of the joints. Arthritis involves inflammation of a joint that is usually accompanied by pain and frequently changes in structure. The invention includes but is not limited to the most common types of arthritis, osteoarthritis and rheumatoid arthritis. Arthritis may also result from or be associated with a number of conditions including infection (infectious arthritis), immunological disturbances and autoimmune disorders (rheumatoid arthritis, juvenile rheumatoid arthritis), trauma, and degenerative joint diseases such as, for example, osteoarthritis.

As used herein, the term “retrovirus” is used in reference to RNA viruses that utilize reverse transcriptase during their replication cycle. The retroviral genomic RNA is converted into double-stranded DNA by reverse transcriptase. This double-stranded DNA form of the virus is capable of being integrated into the chromosome of the infected cell; once integrated, it is referred to as a “provirus.” The provirus serves as a template for RNA polymerase II and directs the expression of RNA molecules which encode the structural proteins and enzymes needed to produce new viral particles. At each end of the provirus are structures called “long terminal repeats” or “LTRs.” LTRs contain numerous regulatory signals, including transcriptional control elements, polyadenylation signals, and sequences needed for replication and integration of the viral genome. LTRs may be several hundred base pairs in length.

As used herein, the term “lentivirus” refers to a group (or genus) of retroviruses that give rise to slowly developing disease. Viruses included within this group include HIV (human immunodeficiency virus; including but not limited to HIV type 1 and HIV type 2), the etiologic agent of the human acquired immunodeficiency syndrome (AIDS); visna-maedi, which causes encephalitis (visna) or pneumonia (maedi) in sheep; the caprine arthritis-encephalitis virus, which causes immune deficiency, arthritis, and encephalopathy in goats; equine infectious anemia virus (EIAV), which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes (i.e., T-cells).

Lentivirus virions have bar-shaped nucleoids and contain genomes that are larger than other retroviruses. Lentiviruses use tRNA^{lys} as primer for negative-strand synthesis, rather than the tRNA^{pro} commonly used by other infectious mammalian retroviruses. The lentiviral genomes exhibit homology with each other, but not with other retroviruses (See, Davis et al. (1990) Microbiology, 4th ed., J.B. Lippincott Co., Philadelphia, Pa., pp. 1123-1151). An important factor in the disease caused by these viruses is the high mutability of the viral genome, which results in the production of mutants capable of evading the host immune response.

As used herein, the term “vector” is used in reference to nucleic acid molecules that transfer nucleic acid (e.g., DNA) segment(s) from one cell to another. For example, vectors include, but are not limited to viral particles, plasmids, transposons, etc.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. In some embodiments, “expression vectors” are used in order to permit pseudotyping of the viral envelope proteins.

The term “lentiviral gene delivery vector” as used herein refers to a vector from which all viral genes have been removed and replaced by a therapeutic gene/cDNA of interest. The viral elements that are retained in the vector include those essential for efficient synthesis and packaging of the viral RNA genome within the viral producer cell (Long Terminal Repeats [LTRs], the packaging signal [psi], and the Rev Responsive Element [RRE]). In addition, viral elements that enable the effective transduction and integration of the viral DNA into the genome of a target cell are also retained in the gene delivery vector (central polypurine tract [cPPT], polypurine tract [ppt]). Finally, regulatory elements that direct high level, long-term expression of the transferred therapeutic gene/cDNA within the transduced target cell are included in the vector (i.e. the elongation factor-1alpha promoter [EF1]).

The term “nucleic acid cassette” as used herein refers to genetic sequences within the vector which can express a RNA, and subsequently a protein. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. In a preferred embodiment of the invention, the nucleic acid cassette contains the sequence of a therapeutic gene used to treat arthritis.

The term “promoter” as used herein refers to a recognition site of a DNA strand to which the RNA polymerase binds. The promoter forms an initiation complex with RNA polymerase to initiate and drive transcriptional activity. The complex can be modified by activating sequences termed “enhancers” or inhibitory sequences termed “silencers”.

The terms “transformation,” “transfection,” and “transduction” refer to introduction of a nucleic acid, e.g., a viral vector, into a recipient cell.

The terms “Pseudotype” or “pseudotyping” as used herein, refer to a virus whose viral envelope proteins have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4+ presenting cells. In a preferred embodiment of the invention, lentiviral envelope proteins are pseudotyped with VSV-G.

As used herein, the term “packaging” refers to the process of sequestering (or packaging) a viral genome inside a protein capsid, whereby a virion particle is formed. This process is also known as encapsidation. As used herein, the term “packaging signal” or “packaging sequence” refers to sequences located within the retroviral genome which are required for insertion of the viral RNA into the viral capsid or particle. Several retroviral vectors use the minimal packaging signal (also referred to as the psi [ψ]sequence) needed for encapsidation of the viral genome. Thus, as used herein, the terms “packaging sequence,” “packaging signal,” “psi” and the symbol “ψ,” are used in reference to the non-coding sequence required for encapsidation of retroviral RNA strands during viral particle formation.

As used herein, the term “packaging cell lines” is used in reference to cell lines that do not contain a packaging signal, but do stably or transiently express viral structural proteins and replication enzymes (e.g., gag, pol and env) which are necessary for the correct packaging of viral particles.

As used herein, the term “replication-defective” refers to virus that is not capable of complete, effective replication such that infective virions are not produced (e.g. replication-defective lentiviral progeny). The term “replication-competent” refers to wild-type virus or mutant virus that is capable of replication, such that viral replication of the virus is capable of producing infective virions (e.g., replication-competent lentiviral progeny).

As used herein, the term “rev” is used in reference to the HIV gene which encodes “Rev,” a protein which interacts with the Rev-response element and helps control viral nucleic acid transport from the nucleus to the cytoplasm. As used herein, the “Rev-response element” or “RRE” refers to the region of viral genome that interacts with Rev.

As used herein, the term “incorporate” refers to uptake or transfer of a vector (e.g., DNA or RNA) into a cell such that the vector can express a therapeutic gene product within the cell. Incorporation may involve, but does not require, integration of the DNA expression vector or episomal replication of the DNA expression vector.

II. Lentiviral Vectors

The present invention provides an improved method for treating arthritis using a lentivirus-based gene delivery system which exhibits sustained, high-level expression of transferred therapeutic genes during in vivo and ex vivo treatment. Lentiviral vectors employed in the gene delivery system are highly efficient at infecting and integrating in a non-toxic manner into the genome of a wide variety of cell types. More particularly, the instant invention provides a recombinant lentivirus capable of infecting non-dividing cells as well as methods and means for making same.

Suitable lentiviral vectors for use in the invention include, but are not limited to, human immunodeficiency virus (e.g., HIV-1, HIV-2), as described in the examples below, feline immunodeficiency virus (FIV), simian immunodeficiency virus (SIV), bovine immunodeficiency virus (BIV), and equine infectious anemia virus (EIAV). These vectors are constructed and engineered using art-recognized techniques to increase their safety for use in therapy and to include suitable expression elements and therapeutic genes, such as those described below, which encode therapeutic proteins for treating arthritis.

In consideration of the potential toxicity of lentiviruses, there are different ways to design the vector in order to increase the safety of the recombinant lentivirus vectors for use as gene transfer vehicles in gene therapy applications. In one embodiment, the vector is made safer by separating the necessary lentiviral genes (e.g., gag and pol) onto separate vectors as described, for example, in U.S. patent application Ser. No. 09/311,684, the contents of which are incorporated by reference herein. Thus, recombinant retrovirus can be constructed in which part of the retroviral coding sequence (gag, pol, env) is replaced by a gene of interest rendering the retrovirus replication defective. The replication defective retrovirus is then packaged into virions through the use of a helper virus or a packaging cell line, by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. In another embodiment, the vector is made safer by replacing certain lentiviral sequences with non-lentiviral sequences. Thus, lentiviral vectors of the present invention may contain partial (e.g., split) gene lentiviral sequences and/or non-lentiviral sequences (e.g., sequences from other retroviruses) as long as its function (e.g., viral titer, infectivity, integration and ability to confer high levels and duration of therapeutic gene expression) are not substantially reduced. Elements which may be cloned into the viral vector include, but are not limited to, promoter, packaging signal, LTR(s), polypurine tracts, RRE, etc.

The infectivity of retroviruses, including lentiviruses, is dependent upon the interaction between glycoproteins displayed on the surface of the viral particle and receptors found on the surface of the target cell. HIV is only able to infect T-cells that display the CD4+ receptor on their cell surfaces. To maximize the infectivity of an HIV-based gene delivery system, the lentivirus is pseudotyped to display a glycoprotein known to bind a wider range of cell type than HIV. In a preferred embodiment of the invention, the recombinant lentivirus is pseudotyped with the vesicular stomatitis virus G coat protein (VSV-G). Pseudotyping with VSV-G increases both the host range and the physical stability of the viral particles, and allows their concentration to very high titers by ultracentrifugation (Naldini et al. (1996), supra; Aiken (1997) J. Virol. 71:5871-5877; Akkina et al., supra; Reiser et al. (1996) Proc. Natl. Acad. Sci. USA 93:15266-15271).

The promoter of the lentiviral vector can be one which is naturally (i.e., as it occurs with a cell in vivo) or non-naturally associated with the 5′ flanking region of a particular gene. Promoters can be derived from eukaryotic genomes, viral genomes, or synthetic sequences. Promoters can be selected to be non-specific (active in all tissues), tissue specific, regulated by natural regulatory processes, regulated by exogenously applied drugs, or regulated by specific physiological states such as those promoters which are activated during an acute phase response or those which are activated only in replicating cells. Non-limiting examples of promoters in the present invention include the retroviral LTR promoter, cytomegalovirus immediate early promoter, SV40 promoter, dihydrofolate reductase promoter. The promoter can also be selected from those shown to specifically express in the select cell types which may be found associated with arthritis.

The lentiviral vector should contain certain elements that will allow for the correct expression of the nucleic acid cassette, i.e. therapeutic gene of interest. One skilled in the art will recognize that the selection of the promoter will depend on the vector, the nucleic acid cassette, the cell type to be targeted, and the desired biological effect. One skilled in the art will also recognize that in the selection of a promoter the parameters can include: achieving sufficiently high levels of gene expression to achieve a physiological effect; maintaining a critical level of gene expression; achieving temporal regulation of gene expression; achieving cell type specific expression; achieving pharmacological, endocrine, paracrine, or autocrine regulation of gene expression; and preventing inappropriate or undesirable levels of expression. Any given set of selection requirements will depend on the conditions but can be readily determined once the specific requirements are determined. Those promoters which naturally occur in the cells comprising the synovia joint, and restrict expression to this site will be preferred.

Standard techniques for the construction of the vectors of the present invention are well-known to those of ordinary skill in the art and can be found in such references as Sambrook et al. (1989) Molecular Cloning: A Laboratory Manual, 2nd Ed. Cold Spring Harbor, N.Y. A variety of strategies are available for ligating fragments of DNA, the choice of which depends on the nature of the termini of the DNA fragments and which choices can be readily made by the skilled artisan.

A variety of therapeutic proteins, to be discussed below, can be encoded by the sequence in a nucleic acid cassette to be expressed in the transformed cells. These proteins can be post-translationally modified to be proteins, glycoproteins, lipoproteins, phosphoproteins, etc. Those proteins which can be expressed may function as intracellular or extracellular structural elements, ligands, hormones, neurotransmitters, growth regulating factors, enzymes, serum proteins, receptors, carriers for small molecular weight compounds, drugs, immunomodulators, oncogenes, tumor suppressors, toxins, tumor antigens. These proteins may have a natural sequence or a mutated sequence to enhance, inhibit, regulate, or eliminate their biological activity. The gene of interest can be obtained for insertion into the viral vector through a variety of techniques known to one of ordinary skill in the art.

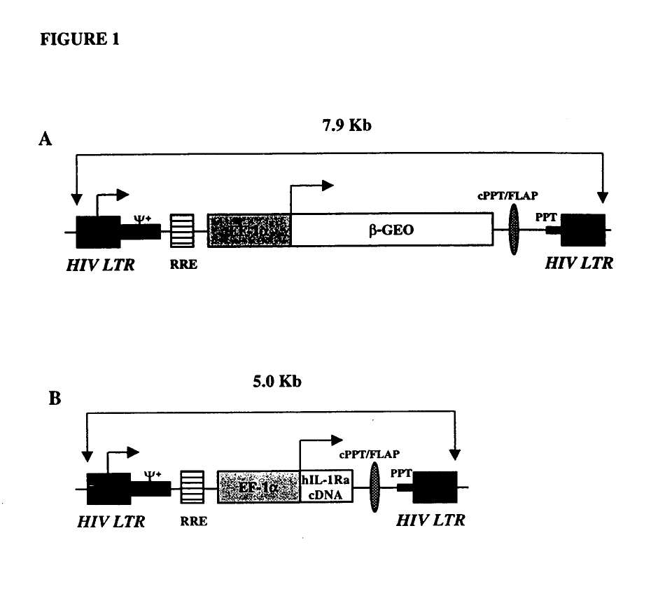

In a preferred embodiment of the invention, the viral vector incorporates the HIV-1 viral backbone, as shown in FIG. 1. This HIV-based recombinant lentiviral vector contains, in a 5′ to 3′ direction, the 5′ flanking HIV LTR, a packaging signal or ψ+, a Rev-response element (RRE), the EF-1α promoter, the therapeutic gene of interest, a central polypurine tract/DNA flap (cPPT/FLAP), a polypurine tract (PPT), and the 3′ flanking HIV LTR. cDNA of the therapeutic gene of interest is amplified by PCR from an appropriate library. The gene is cloned into a plasmid, such as pBluescript II KS (+) (Stratagene), containing a desired promoter, such as the human EF-1α promoter. Following restriction enzyme digestion, or other method known by one skilled in the art to obtain a desired DNA sequence, the nucleic acid cassette containing the promoter and therapeutic gene of interest is then inserted into an appropriate cloning site of the HIV-1 viral vector, as shown in FIG. 1.

A major prerequisite for the use of viruses as gene delivery vectors is to ensure the safety of their use, particularly with regard to the possibility of the spread of wild-type virus in the cell population. The development packaging cell lines, which produce only replication-defective retroviruses, has increased the utility of retroviruses for gene therapy, and defective retroviruses are well characterized for use in gene transfer for gene therapy purposes (for a review see Miller, A. D. (1990) Blood 76:271). Accordingly, in one embodiment of the invention, packaging cell lines can be used to propagate lentiviral vectors of the invention to increase the titer of the vector virus. The use of packaging cell lines is also considered a safe way to propagate the virus, as use of the system reduces the likelihood that recombination will occur to generate wild-type virus. In addition, to reduce toxicity to cells that caused by expression of packaging proteins, packaging systems can be use in which the plasmids encoding the packaging functions of the virus are only transiently transfected by, for example, chemical means.

The step of facilitating the production of infectious viral particles in the cells may be carried out using conventional techniques, such as standard cell culture growth techniques. If desired by the skilled artisan, lentiviral stock solutions may be prepared using the vectors and methods of the present invention. Methods of preparing viral stock solutions are known in the art and are illustrated by, e.g., Y. Soneoka et al. (1995) Nucl. Acids Res. 23:628-633, and N. R. Landau et al. (1992) J. Virol. 66:5110-5113. In a method of producing a stock solution in the present invention, lentiviral-permissive cells (referred to herein as producer cells) are transfected with the vector system of the present invention. The cells are then grown under suitable cell culture conditions, and the lentiviral particles collected from either the cells themselves or from the cell media as described above. Suitable producer cell lines include, but are not limited to, the human embryonic kidney cell line 293, the equine dermis cell line NBL-6, and the canine fetal thymus cell line Cf2TH.

The step of collecting the infectious virus particles also can be carried out using conventional techniques. For example, the infectious particles can be collected by cell lysis, or collection of the supernatant of the cell culture, as is known in the art. Optionally, the collected virus particles may be purified if desired. Suitable purification techniques are well known to those skilled in the art.

Other methods relating to the use of viral vectors in gene therapy can be found in, e.g., Kay, M. A. (1997) Chest 111(6 Supp.):138S-142S; Ferry, N. and Heard, J. M. (1998) Hum. Gene Ther. 9:1975-81; Shiratory, Y. et al. (1999) Liver 19:265-74; Oka, K. et al. (2000) Curr. Opin. Lipidol. 11: 179-86; Thule, P. M. and Liu, J. M. (2000) Gene Ther. 7:1744-52; Yang, N. S. (1992) Crit. Rev. Biotechnol. 12:335-56; Alt, M. (1995) J. Hepatol. 23:746-58; Brody, S. L. and Crystal, R. G. (1994) Ann. N.Y. Acad. Sci. 716:90-101; Strayer, D. S. (1999) Expert Opin. Investig. Drugs 8:2159-2172; Smith-Arica, J. R. and Bartlett, J. S. (2001) Curr. Cardiol. Rep. 3:43-49; and Lee, H. C. et al. (2000) Nature 408:483-8.

III. Therapeutic Genes

Suitable therapeutic genes for use in the present invention include genes which encode proteins which are useful in treating arthritis. As will be appreciated by one skilled in the art, the nucleotide sequence of the inserted therapeutic gene may be the entire gene sequence or any functional portion thereof (e.g., which, when expressed, encodes a protein or peptide capable of treating arthritis). Representative examples of genes which have been proven effective at treating arthritis include but are not limited to the following: soluble IL-1 receptors, antagonists of the IL-1 receptors, soluble TNF-α receptors, fibronectin and fibronectin fragments, TGF-β family members, IGF-1, LIF, BMP-2, BMP-7, plasminogen activators, plasminogen inhibitors, MMPs, TIMPs, Indian Hedgehog, parathyroid hormone-related protein, IL-4, IL-10, IL-11, IL-13, hyaluronan synthase, and PDGF.

Interleukin-1 (IL-1) Receptors and Antagonists of the Receptor

It is well accepted that IL-1beta plays a pivotal role in the progression of OA (Pelletier et al. (1997) Arthritis. Rheum. 40:1012; Van de Loo, et al. (1995) Arthritis Rheum. 38:164; Goldring (1999) Connect. Tissue Res. 40:1). This factor is known to stimulate the production and release of a variety of inflammatory factors such as IL-6, IL-8, LIF and prostaglandin (PG) E2 from both articular chondrocytes and synovial fibroblasts (Martel-Pelletier et al. (1999) supra; Lotz et al. (1992) J. Clin. Invest. 90:888; Chevalier et al. (1997) Biomed. Pharmacother. 51:58; Amin et al. (1999) Curr. Opin. Rheumatol. 11:202). In addition, destruction of the articular cartilage is enhanced through the upregulation of a number of MMPs (including MMP-1, MMP-2, MMP-3, MMP-9 and MMP-13) and the suppression of proteoglycan, collagen and TIMP synthesis (Chevalier et al., supra; Studer et al. (1999) Osteoarthritis Cartilage 7:377).

The biological activity of IL-1beta is transduced through the IL-1 receptor (IL-1R) of which there are two types; type I and type II (Slack et al. (1994) J. Biol. Chem. 268:2513). In cells of the articular cartilage the type I receptor, which has a greater affinity for IL-1beta as compared to the type II receptor, appears to be responsible for signal transduction (Arend (1993) Adv. Immunol. 54:167; Martel-Pelletier et al. (1992) Arthritis Rheum. 35:530; Sadouk et al. (1995) Lab. Invest. 73:347). It is unclear whether the type II receptor mediates IL-1 signaling in these cells or serves rather as a competitive inhibitor of IL-1 binding to the type I receptor. Both types of receptors, however, are actively shed from the surface of articular tissue cells as soluble receptors (sIL-1R) and can act as antagonists of IL-1 signal transduction (Martel-Pelletier et al. (1999), supra). Recombinant soluble type II IL-1R was shown to significantly inhibit disease progression in a mouse model of arthritis (Bessis et al., supra).

IL-1 signaling is also regulated through the production of an IL-1R antagonist (IL-1Ra), a naturally occurring glycoprotein which is released primarily by macrophages (Martel-Pelletier et al. (1999), supra). IL-1Ra competes with IL-1 for binding of the IL-1R although it does not transduce any biological signals following receptor binding (Martel-Pelletier et al. (1999), supra). Importantly, IL-1Ra has been shown to block many of the catabolic effects of IL-1beta including the production of inflammatory molecules and MMPs as well as the suppression of extracellular matrix molecule and TIMP synthesis (Martel-Pelletier et al. (1999), supra). In animal models of OA, recombinant IL-1Ra reduced cartilage degradation, MMP production and the progression of cartilage lesions (Caron et al., supra). In clinical trials, arthritis patients who received recombinant IL-1Ra subcutaneously showed a significant slowing of radiographic progression of the disease at 24 weeks (Bresnihan et al., supra). The efficacy of using the IL-1Ra cDNA for gene therapy has also been investigated. Introduction of the IL-1Ra cDNA into animal synovial fibroblasts ex vivo significantly reduced the progression of joint remodeling following transplantation in a dog model of OA (Pelletier et al., supra). Moreover, transfer of the human IL-1Ra cDNA into human chondrocytes was shown to protect OA cartilage explants from IL-1 induced degradation in vitro (Baragi et al., supra).

There is evidence to suggest that combining IL-1Ra and sIL-1R together can have additive beneficial effects. This is dependent, however, upon the type of sIL-1R used as the affinity of the soluble receptors for IL-1beta and the IL-1Ra differs. While the type I sIL-1R binds the IL-1Ra with high affinity as compared to IL-1beta, the type II sIL-1R binds IL-1 beta more readily than IL-1Ra (Sadouk et al., supra; Bell et al. (2000) J. Rheumatol. 27:332; Dinarello (1996) Blood 87:2095; Svenson et al. (1993) Cytokine 5:427). Thus, in the presence of type II sIL-1R the inhibitory effects of IL-1Ra are additive (Martel-Pelletier et al. (1999), supra).

Accordingly, in one embodiment of the present invention, high, sustained levels of soluble type II IL-1 receptor in combination with the IL-1 receptor antagonist are used to treat arthritis by way of the lentiviral-based gene delivery system described herein.

TNF-α Receptors

Similar to IL-1beta, TNF-α is believed to play a direct and pivotal role in the initiation/progression of OA. Transgenic mice engineered to constitutively express human TNF-α spontaneously develop polyarthritis (Meyer et al. (2000) Presse. Med. 29:463). TNF-α, secreted from macrophages and articular chondrocytes, acts through 2 different TNF-α receptors (TNF-R55 and TNF-R75) expressed on the surface of articular chondrocytes and synovial fibroblasts (Martel-Pelletier et al. (1999), supra). Also similar to IL-1, TNF-α has pleiotropic effects which include an upregulation of type I and type II IL-1 receptors, TNF-α receptors 55 and 75, IL-6 receptor, IL-1beta, TNF-α, LIF, IL-8, prostaglandin E2 and IL-6 (Martel-Pelletier et al. (1999), supra); Shlopov et al. (2000) Arthritis Rheumatol. 43:195; Larrick et al. (1988) Pharmaceut. Res. 5:129; Westacott et al. (1996) Arthritis Rheumatol. 25:254; Alaaeddine et al. (1997) J. Rheumatol. 24:1985; Alaaeddine et al. (1999) Arthritis Rheumatol. 42:710). In addition, TNF-α stimulates the production and secretion MMP-1, MMP-8 and MMP-13 from articular chondrocytes (Shlopov et al., supra).

Soluble forms of TNF-R55 and TNF-R75 are actively produced and shed from synovial fibroblasts and chondrocytes and play an important role in regulating TNF-α activity by sequestering the protein and preventing it from transducing its signal (Larrick et al., supra; Westacott et al., supra; Alaaeddine et al. (1997), supra; Alaaeddine et al (1999), supra). These soluble receptors have been shown to be transiently effective in preventing the progression of arthritis in both animal models (Ghivizzani et al. (1998), supra) and in clinical trials (McKay et al., supra; Moreland et al. (1999), supra; Moreland et al. (1997), supra).

Accordingly, in another embodiment of the present invention, soluble TNF-α receptors are used to treat arthritis by way of the lentiviral-based gene delivery system described herein.

Fibronectin and Fibronectin Fragments

Fibronectin is one of the major components of the extracellular matrix of articular cartilage and plays an important role in the maintenance of cartilage homeostasis. Fibronectin fragments, such as those produced as the result of MMP activity in OA enhance the levels of catabolic cytokines (IL-1beta, TNF-α and IL-6), upregulate the expression of a variety of MMPs, enhance the degradation and loss of proteoglycans from the cartilage and temporarily suppress the biosynthesis of new extracellular matrix molecules (Homandberg (1999) Frontiers in Bioscience 4:713). These activities are apparently the result of interaction of the fibronectin fragments with the alpha5beta1 integrin receptor since the binding of anti-alpha5beta1 integrin antibodies to this receptor produces the same effect (Homandberg, supra).

Of importance, synthetic proteins which antagonize the binding of fibronectin fragments to the alpha5beta1 integrin receptor are known (Homandberg, supra). Although these proteins bind the alpha5beta1 receptor, they do not induce catabolic signaling events and can block the binding and subsequent signaling of fibronectin fragments.

Accordingly, in another embodiment of the present invention, proteins which antagonize the binding of fibronectin fragments to the alpha5beta1 integrin receptor are used to treat arthritis by way of the lentiviral-based gene delivery system described herein.

Transforming Growth Factor-β

TGF-β is desirable as a therapeutic agent due to its pleiotropic effects upon articular chondrocytes. TGF-β blocks the degradation of the articular cartilage by down regulating the production of MMP-1, MMP-13, IL-1 receptors type I and II, TNF-α receptors 55 and 75, IL-1beta, TNF-α and IL-6 (Shlopov et al., supra) as well as upregulating TIMP-1 and -3 (Su et al. (1996) DNA Cell Biol. 15:1039; Su et al. (1999) Rheumatol. Int. 18:183; Frenkel et al. (2000) Plast. Reconstr. Sur. 105:980). Moreover, TGF-β also stimulates the regeneration of articular cartilage by stimulating the synthesis of a variety of matrix molecules including proteoglycans (Lafeber et al. (1997) J. Rheumatol. 24:536; Van Beuningen et al. (1994) Lab. Invest. 25:613), fibronectin (Sarkissan et al. (1998) J. Rheumatol. 26:613) and collagen (Mansell et al. (1998) J. Clin. Invest. 101:1596; Galera et al. (1992) J. Cell Physiol. 152:596).

Accordingly, in another embodiment of the present invention, TGF-β is used to treat arthritis by way of the lentiviral-based gene delivery system described herein.

Insulin-Like Growth Factor-1 (IGF-1):

IGF-1 is the major anabolic factor in articular cartilage (Olney et al. (1996) J. Clin. Endocrinol. Metab. 81:1096). IGF-1 blocks the catabolic effects of IL-1beta and TNF-α, stimulates the synthesis of a variety of extracellular matrix molecules and is mitogenic for articular chondrocytes (Olney et al., supra; Trippel et al. (1995) J. Rheum. Suppl. 45:129). The activity of IGF in articular cartilage is modulated by a family of at least 6 proteins called IGF binding proteins (IGFBP). These binding proteins have a high affinity for IGF and prevent its interaction with the IGF receptor (Olney et al., supra). Interestingly, articular cartilage from OA patients shows a significant increase in both IGF and several IGFBPs (Olney et al., supra; Fernihough et al. (1996) Arthritis Rheum. 39:1556). However, the levels of IGFBPs are elevated several fold over IGF resulting in an overall suppression of its anabolic activity (Olney et al., supra). For example, while levels of IGF mRNA in OA chondrocytes were increased 3.5-fold over normal, levels of IGFBP-3 and IGFBP-5 were increased 24 and 16-fold over normal respectively (Olney et al., supra).

Accordingly, in another embodiment of the present invention, IGF-1 protein is used to treat arthritis (by concomitantly increasing IGFBP levels) by way of the lentiviral-based gene delivery system described herein.

Leukemia Inhibitory Factor (LIF) and its Binding Protein

Both articular chondrocytes and synovial fibroblasts produce LIF in response to IL-1beta or TNF-α (Lotz et al., supra; Ishimi et al. (1992) J. Cell Physiol. 152:71; Hui et al. (1998) Cytokine 10:220; Campbell et al. (1993) Arthritis Rheum. 36:790; Hamilton et al. (1993) J. Immunol. 150:1496). The generated LIF reinforces the catabolic effects of IL-1beta and TNF-α by stimulating the synthesis of more IL-1beta and TNF-α from articular tissue, thereby creating a positive feedback loop (Villiger et al. (1993) J. Clin. Invest. 91:1575). In additional, LIF also causes the breakdown of articular cartilage by stimulating the production of MMP-1 and MMP-3, and suppressing the synthesis of cartilage proteoglycans (Lotz et al., supra; Hui et al., supra).

LIF binding protein (LBP), a naturally occurring form of soluble LIF receptor alpha (Hui et al., supra; Bell et al. (1997) J. Rheumatol. 24:2394), has been shown to effectively prevent the effects of LIF induced proteoglycan catabolism both in pig articular cartilage explants ex vivo (Bell et al. (2000), supra) and in goat radiocarpal joints in vivo (Bell et al. (1997), supra).

Accordingly, in another embodiment of the present invention, LBP either alone or in combination with other therapeutic proteins is used to treat arthritis by way of the lentiviral-based gene delivery system described herein.

BMP-2 and -7

Similar to IGF-1 and TGF-β, BMP-2 and BMP-7 have been shown to have a beneficial effect upon cartilage metabolism by stimulating, from chondrocytes, the synthesis of a variety of extracellular matrix molecules including proteoglycan, aggrecan and collagen Type II (Smith et al., supra; Sailor et al. (1996) J. Orthop. Res. 14:937; Van Susante et al. (2000) J. Orthop. Res. 18:68; Flechtenmacher et al. (1996) Arthritis Rheum. 39:1896) and increasing the levels of TIMP expression (Frenkel et al., supra). Moreover, BMP-2 and -7 can block the catabolic effects of IL-1beta (Smith et al., supra) and fibronectin fragments (Koepp et al. (1999) Inflamm. Res. 47:1).

Accordingly, in another embodiment of the present invention, BMP-2 and BMP-7 either alone or in combination with IGF-1 and/or TGF-β are used to treat arthritis by way of the lentiviral-based gene delivery system described herein.

Plasminogen Activators and their Inhibitors

Plasminogen plays an important role in cartilage catabolism. MMPs generated by chondrocyte and synovial fibroblasts in response to catabolic factors such as IL-1beta or TNF-α are synthesized as latent proenzymes and must first undergo proteolytic processing prior to becoming active. One such activating pathway involves the action of plasmin which is generated from plasminogen by urokinase plasminogen activator (uPA). Urokinase plasminogen activator is produced by articular chondrocytes (Martel-Pelletier et al. (1991) J. Rheumatol. 18:1863) and is present in high levels in OA joint tissue (Pelletier et al. (1990) Arthritis Rheum. 33:1466). However, the activity of this enzyme can be potently repressed by plasminogen activator inhibitor (PAI), of which there are two forms, PAI-1 and PAI-2.

Accordingly, in another embodiment of the present invention, plasminogen activators are used to treat arthritis by way of the lentiviral-based gene delivery system described herein.

MMPs and TIMPs

It is clear that MMPs play a direct and predominant role in the destruction of the articular cartilage in OA. A number of MMPs such as MMP-1 (collagenase), MMP-3 (stromelysin), MMP-2 and MMP-9 (gelatinases) as well as MMP-8 and MMP-13 (collagenases) are upregulated in osteoarthritic joints (Yoshihara et al. (2000) Ann. Rheum. Dis. 59:455; Shlopov et al. (1997) Rheum. 40:2065). Interestingly, several TIMPs including TIMP-1 and -2 are also upregulated in OA (Lohmander et al. (1994) J. Orthop. Res. 12:21; Zafarullah et al. (1996) J. Cell. Biochem. 60:211; Martel-Pelletier et al. (1994) J. Lab. Invest. 70:807). Although both MMPs and TIMPs are elevated in OA, the progressive destruction of the articular cartilage occurs as a result of a gross imbalance in the levels of these factors. Several groups have demonstrated a large molar excess of MMPs compared to TIMPs in OA (Su et al. (1999), supra; Lohmander et al. (1993) J. Rheumatol. 20:1362; Dean et al. (1989) J. Clin. Invest. 84:678; Woessner et al. (1991) Rheumatol. Supl. 27:99; Nguyen et al. (1992) J. Clin. Invest. 89:1189). For example, TIMP-1 was found in normal synovial fluid at a 2-fold molar excess over MMP-3 (Lohmander et al. (1993), supra). However, MMP-3 levels were 1.5 to 2.5-fold greater than TIMP-1 levels in patients who had suffered an injury to either their cruciate ligament or meniscus 3 (Lohmander et al. (1993), supra).

In addition to directly inhibiting the effects of MMPs, some TIMPs can also effect the production of catabolic factors such as TNF-α. TNF-α Converting Enzyme (TACE) is a cell surface bound metalloprotease which is required for the processing and release of TNF-α from the surface of the macrophages and articular chondrocytes. Several recent studies have shown that the synthesis of TNF-α can be suppressed by TIMP-3, an inhibitor of TACE (Amour et al. (1998) FEBS Letters 435:39; Amin et al. (1999) Osteoarthritis Cartilage 7:392).

Accordingly, in another embodiment of the present invention, local concentrations of TIMPs within arthritic joints are increased to levels equal to or greater than MMPs to treat arthritis by way of the lentiviral-based gene delivery system described herein.

Indian Hedgehog and Parathyroid Hormone-Related Protein

Indian hedgehog (Ihh) is a secreted protein produced by chondrocytes that are committed to becoming hypertrophic. Ihh induces the synthesis of a second factor called parathyroid hormone-related protein (PTHrP) which binds to its receptor on prehypertrophic chondrocytes to inhibit chondrocyte differentiation (Vortkamp et al. (1996) Science 273:613). Therefore, PTHrP mediates the effects of Ihh through the formation of a negative feedback loop that regulates the rate of chondrocyte differentiation. Moreover, Ihh has been reported to upregulate the expression of BMP-2 (Pathi et al. (1999) Dev. Biol. 209:239).

Accordingly, in another embodiment of the present invention, Ihh or PTHrP are used to counteract the high degree of chondrocyte apoptosis observed in OA by way of the lentiviral-based gene delivery system described herein.

Interleukins-4, -10, -11, and -13

IL-4, IL-10, IL-11 and IL-13 are present in elevated levels in the synovial fluid of OA patients (Martel-Pelletier, et al. (1999), supra) and are potentially very useful for the treatment of OA. All of these cytokines possess anti-inflammatory properties which include decreased production of IL-1beta, TNF-α, prostaglandin E2 and MMPs as well as the upregulation of IL-1R antagonist and TIMP-1 (Alaaeddine et al. (1999), supra; Essner et al. (1989) J. Immunol. 142:3957; Shingu et al. (1995) Br. J. Rheumatol. 34:101; Donnelly et al. (1990) J. Immunol. 145:569; Vannier et al. (1992) Proc. Natl. Acad. Sci. USA, 89:4076; Hart et al. (1995) Immunol. 84:536).

Accordingly, in another embodiment of the present invention, in vivo expression of IL-4, IL-10, IL-11 and IL-13 is used to treat arthritis by way of the lentiviral-based gene delivery system described herein.

Hyaluronan Synthase

As previously mentioned, intra-articular injections of hyaluronan is one of the current treatments for OA. The beneficial effects of hyaluronan injections are most likely due to its ability to downregulate the production of MMP-3 and IL-1beta (Takehashi et al. (1999) Osteoarthritis Cartilage 7:182) and stimulate proteoglycan synthesis (Han et al. (1999) Nagoya J. Med. Sci. 62:115).

Accordingly, in another embodiment of the present invention, in vivo expression of hyaluronan synthase in articular chondrocytes and/or synovial fibroblasts is used treat arthritis by way of the lentiviral-based gene delivery system described herein.

Platelet Derived Growth Factors (PDGF)

PDGF-BB has been reported to stimulate the synthesis of fibronectin from synovial fibroblasts (Trippel et al., supra).

Accordingly, in another embodiment of the present invention, in vivo expression of PDGF-BB, or a related PDGF (e.g., PDGF-AA or PDGF-AB) is used treat arthritis by way of the lentiviral-based gene delivery system described herein.

IV. Therapeutic Uses of Lentiviral Vectors

Administration of Lentiviral Vectors

The lentiviral vectors described above can be administered in vivo to subjects by any suitable route, as is well known in the art. The term “administration” refers to the route of introduction of a formulated vector into the body. For example, administration may be intravenous, intramuscular, topical, oral, or by gene gun or hypospray instrumentation. Thus, administration can be direct to a target tissue or through systemic delivery. Administration directly to the target tissue can involve needle injection, hypospray, electroporation, or the gene gun. See, e.g., WO 93/18759, hereby incorporated by reference herein. In a preferred embodiment, administration is achieved by direct injection to a target tissue, such as the synovial lining of the joints of a subject suffering from arthritis.

Alternatively, the lentiviral vectors of the invention can be administered ex vivo or in vitro to cells or tissues using standard transfection techniques well known in the art.

As used herein “pharmaceutically acceptable carrier” includes any and all solvents, dispersion media, coatings, antibacterial and antifungal agents, isotonic and absorption delaying agents, and the like that are physiologically compatible. In one embodiment, the carrier is suitable for parenteral administration. Preferably, the carrier is suitable for administration directly into an affected joint. The carrier can be suitable for intravenous, intraperitoneal or intramuscular administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Another aspect of the invention pertains to pharmaceutical compositions of the lentiviral vectors of the invention. In one embodiment, the composition includes a lentiviral vector in a therapeutically effective amount sufficient to treat or prevent (e.g. ameliorate the symptoms of arthritis), and a pharmaceutically acceptable carrier. A “therapeutically effective amount” refers to an amount effective, at dosages and for periods of time necessary, to achieve the desired therapeutic result, such as treatment or prevention of arthritis. A therapeutically effective amount of lentiviral vector may vary according to factors such as the disease state, age, sex, and weight of the individual, and the ability of the lentiviral vector to elicit a desired response in the individual. Dosage regimens may be adjusted to provide the optimum therapeutic response. A therapeutically effective amount is also one in which any toxic or detrimental effects of the lentiviral vector are outweighed by the therapeutically beneficial effects. The potential toxicity of the lentiviral vectors of the invention can be assayed using cell-based assays or art recognized animal models and a therapeutically effective modulator can be selected which does not exhibit significant toxicity. In a preferred embodiment, a therapeutically effective amount of a lentiviral vector is sufficient to treat arthritis.

Sterile injectable solutions can be prepared by incorporating lentiviral vector in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

It is to be noted that dosage values may vary with the severity of the condition to be alleviated. It is to be further understood that for any particular subject, specific dosage regimens can be adjusted over time according to the individual need and the professional judgment of the person administering or supervising the administration of the compositions, and that dosage ranges set forth herein are exemplary only and are not intended to limit the scope or practice of the claimed composition.

The amount of lentiviral vector in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

In Vivo Uses of Lentiviral Vectors

A major advantage of lentiviral vectors is that they are capable of integrating into the genome of a host cell and, therefore, enable long term expression of therapeutic proteins. Lentiviral vectors have been successfully used to deliver exogenous genes both in vitro and in vivo to a large variety of cell populations in several species, including neurons of the central nervous system (Naldini et al. (1996) Proc. Natl. Acad. Sci. USA 93:11382-11388), retinal cells (Miyoshi et al. (1997) Proc. Natl. Acad. Sci. USA 94:10319-10323), and pancreatic cells (Giannokakis et al. (1999) Gene Ther. 6:1545-1551).

Lentiviral vectors can be used as highly efficient vehicles for direct gene transfer to tissues, including the synovium. In particular, as described herein and as exemplified below, the lentiviral vectors of the present invention have the capacity to infect and genetically modify synovial cell cultures from a variety of species, including humans. Furthermore, following intra-articular injection, these lentiviral vectors are capable of delivering exogenous therapeutic genes to the joints of rats and achieving high, sustained levels of transgene expression. The instant invention describes a method for treating arthritis by delivering to a subject in vivo, a therapeutic gene using a lentiviral gene delivery system such that the gene is expressed at sufficient levels and for a sufficient period. In one embodiment of the invention, lentiviral vectors mediate transgene expression that is four-fold that as compared to adenoviral vector mediated expression.

In a preferred embodiment of the invention, the lentiviral vector is selected from the group consisting of HIV, FIV, SIV, BIV, and EIAV. Virus containing lentiviral vectors used for in vivo treatment in a subject suffering from arthritis can be produced using packaging cell lines in order to increase the safety of the gene delivery system. Administration of the lentiviral vector containing the therapeutic gene can be through any of the methods described above, but is preferably through direct injection into an affected joint of the subject.

Ex Vivo Uses of the Lentiviral Vector

Lentiviral vectors containing therapeutic genes also can be transiently transfected into cells for ex vivo modification. Transduced cells which express the therapeutic gene at sufficient levels can then be isolated and administered to a subject for the treatment of arthritis. In one embodiment of the invention, the lentiviral gene delivery vector is selected from the group consisting of HIV, SIV, FIV, BIV, and EIAV. In a further embodiment of the invention, the transduced cells are autologous wherein the cells can be, but are not limited to, bone marrow cells, mesenchymal stem cells obtained from adipose tissue, or synovial fibroblast or chondrocytes. In another embodiment of the invention, the cells to be administered which contain the lentiviral vector are non-autologous, including both allogeneic and xenogeneic cells. These cells can be from a cell line or alternatively can also be primary cells derived from human or animal sources.

In summary, the viral vectors of the present invention can be used to stably transduce either dividing or non-dividing cells, and stably express a therapeutic gene. Using this vector system, it is possible to introduce into dividing or non-dividing cells, genes which encode proteins that can affect the physiology of cells within arthritic joints. Furthermore, the lentiviral vectors of the invention are highly efficient vehicles for direct gene transfer to synovium. The vectors of the present invention can thus be useful in gene therapy for arthritis.

### EXAMPLES

In the following examples high-titer VSV-G pseudotyped, HIV-1-based lentiviral vectors (FIG. 1) were evaluated for their ability to deliver exogenous genes to articular tissues in situ. These examples demonstrate that, following direct intra-articular injection, lentiviral vectors efficiently transduce synovial cells, resulting in high levels of transgene expression. Moreover, in athymic animals, intra-articular, lentivirus-mediated transgene expression is sustained for at least 42 days following delivery. These examples demonstrate that lentiviral vectors have the capacity to infect and genetically modify synovial cell cultures from a variety of species, including human, and that following intra-articular injection, they are capable of delivering exogenous genes to the joints of rats and achieving high, sustained levels of transgene expression. Furthermore, these examples demonstrate that lentiviral delivered hIL-1Ra can prevent both local and systemic sequelae of highly destructive experimental arthritis driven by synovial expression of IL-1.

Materials and Methods

Lentiviral and Adenoviral Vector Production

The HIV-1 viral backbone, extended packaging signal, central polypurine tract/FLAP and the rev-responsive element were obtained from the recombinant clone pNL4-3 (genbank accession # M19921). The β-GEO gene was constructed by fusing the coding regions of the β-galactosidase (Ory et al. (1996) Proc. Natl. Acad. Sci. USA 93:11400-11406) and neomycin resistance genes (Stratagene, La Jolla, Calif.) with an oligonucleotide. In addition, to achieve nuclear accumulation of β-galactosidase staining and allow better differentiation between transduced and background, non-transduced cells, the β-galactosidase gene was engineered to contain a nuclear localization signal fused in frame to the β-galactosidase sequence (Ory et al., supra). The human IL-1Ra cDNA was amplified by PCR from a human monocyte cDNA library (Bandara et al. (1993), supra). The β-GEO gene, the hIL-1Ra cDNA, or the firefly luciferase gene was cloned into pBluescript II KS (+) (Stratagene, La Jolla, Calif.) 3′ of the human EF-1α promoter following digestion with Nco I and BamHI. Cassettes were then inserted into the BamHI site of the HIV-1 viral backbone (The DNA sequence of the vector will be provided upon request). Virus stocks were generated by transient transfection of 293T cells with the recombinant lentiviral vector combined with pcRevCMV (Malim et al. (1988) Nature 335:181-183), pHCMV-G (Yee et al. (1994) Proc. Natl. Acad. Sci. USA 91:9564-9568) and a CMV-Tat expression plasmid derived from pNL4-3. Plasmid constructs (FIG. 1) were transfected into 293T cells using CaPO₄ precipitation. Viral supernatants were collected 48 hours later, filtered through 0.45 μm filters and concentrated 500-fold by ultracentrifugation at 25,000 rpm for 90 minutes at 4° C. Titers were estimated by Southern blot analysis, using a radiolabeled fragment of the human IL-1Ra cDNA as a probe (Pawliuk et al. (1994) Blood 84:2868-2877). Quantitation of vector copy number was achieved by densitometry using a PhosphorImager with ImageQua™ software (Molecular Dynamics, Sunnyvale, Calif.) and compared to an NIH 3T3 cell line known to contain one copy of recombinant IL-1Ra provirus. Each virus preparation was assessed for the presence of Replication Competent Retrovirus (RCR) using the neomycin resistance mobilization assay as described (Pawliuk et al., supra). Prior to injection, efficient and stable transfer of the hIL-1Ra cDNA to target cells was verified by Southern blot analysis of genomic DNA from NIH3T3 cells following exposure to recombinant lentivirus (data not shown). Virus titers were estimated to be approximately 1×10⁹ iu/ml.

The adenoviral vector (Ad.LacZ) used in this work originated from replication-deficient type 5 adenovirus lacking E1 and E3 loci. The gene encoding the β-galactosidase of E. coli was inserted in place of the E1 region, with expression driven by the human cytomegalovirus early promoter (Yeh et al. (1997) Faseb J. 11:615-623). High-titer suspensions of recombinant adenovirus were prepared by amplification in 293 cells, and purified using three consecutive CsCl gradients by established methods (Palmer et al. (In Press) Methods Mol. Biol.) Titers were determined by optical density at 260 nm and standard plaque assay

Tissue Culture and In Vitro Transduction

Rat and human chondrocytes were cultured in Ham's F12 medium (Gibco-BRL). Rat and human synoviocytes, a murine fibroblast cell line, 3T3, and a rabbit synovial cell line, HIG-82 (Georgescu et al. (1988) In vitro Cell. Dev. Biol. 24:1015-1022), were cultured in Dulbecco's Modified Eagle medium (Gibco-BRL). All cells were grown to approximately 75% confluence in 24-well plates containing 1 ml of corresponding medium supplemented with 10% fetal bovine serum and 1% penicillin/streptomycin (Gibco-BRL). For the lacZ experiments, the cells were transduced by incubation overnight at 37° C. with 5×10⁷ infectious units (iu) of lentivirus in 700 μl of corresponding serum-free medium containing 7 μg/ml protamine sulfate (Sigma, St Louis, Mo.). Afterwards, the medium was replaced, and the cells returned to the incubator for 48 hours. Cells were then fixed in 4% paraformaldehyde and stained for α-galactosidase activity in the presence of 1 mg/ml X-Gal in 2.5 mM K₄Fe(CN)₆, 2.5 mM K₃Fe(CN)₆, 50 mM Tris-HCl pH 8.0 for 4 hours at 37° C. For the in vitro characterization of the hIL-1Ra lentivirus, 10⁵ rat synovial cells were incubated overnight in 1 ml of medium with 10-fold dilutions of lentivirus, starting with 5×10⁶ iu (MOI from 50 to 5×10⁻⁴). The medium was harvested 24 hours later and the hIL-1Ra content measured by ELISA.

Experimental Animals and Intra-Articular Injection

Experiments were performed on Wistar male rats weighing 150-175 g (Charles River Laboratories, Wilmington, Mass.), and 6-7 week old athymic nude rats (Harlan, Indianapolis, Ind.) housed two per cage with free access to standard laboratory food and water. All animal procedures were approved by the Harvard Medical Area Standing Committee on Animals. A total of 5×10⁷ iu of either hIL-1Ra or control virus was suspended in 50 μl of phosphate buffered saline and injected into the joint space of the knee through the infrapatellar ligament. Animals were sacrificed by CO₂ asphyxiation.

Biological Analyses

Following sacrifice of the animals, the skin was removed from the legs, and the knees were dissected using a scalpel. Incisions were made along the lateral and medial sides of the harvested knees, and the capsule attached to the patella was folded back, exposing the articular surfaces. The lateral collateral, and anterior and posterior cruciate ligaments were then transected to allow exposure of the entire joint capsule. At this time the joints were either stained for β-galactosidase activity or washed with saline, placed in 24-well dishes with 1 ml of complete DMEM and cultured for 24 hours at 37° C., 5% CO₂. For the hIL-1Ra experiments, the heart, liver, lung, spleen, and gonads of the animals were also harvested and placed in saline solution. Blood samples were collected by cardiac puncture and centrifuged; serum was collected and stored at −20° C. until testing. To measure the synthesis of hIL-1Ra in the harvested organs, an approximate 100 mg portion from each tissue was minced with a scalpel and placed in 1 ml of culture medium for 24 hours. The conditioned media from the knee and organ cultures were then removed and stored at −20° C. until testing. hIL-1Ra concentrations were measured using ELISA kits from R&D Systems (Minneapolis, Minn.) as directed by the supplier.

RT-PCR analyses were performed on total RNA extracted from the harvested organs. Briefly, the organs were homogenized in the presence of Trizol solution and extracted with chloroform. RNA was then precipitated with isopropanol. One microgram of total RNA was reverse transcribed using random hexanucleotide primers (Gibco-BRL, Rockville, Md.). For PCR amplification, primer pairs were specific for detection of human IL-1Ra. The sensitivity of the assay is <1 positive cell in one thousand.

To determine luciferase expression biodistribution, rats were sacrificed 2, 5 or 10 days following intra-articular injection of luciferase lentivirus. The harvested tissues (knees and organs) were dissected, mixed with 2 ml of Gey's balanced salt solution and homogenized using a motorized homogenizer. Following incubation for 2-3 minutes at room temperature of the homogenate with an equal volume of lysis buffer (Bright-Glo™ Luciferase Assay System; Promega, Madison, Wis.), the homogenate was centrifuged at low speed in a table-top clinical centrifuge, and luciferase activity in 500 μl of the supernatant measured in a luminometer.

For histological analysis, tissues harvested from dissected knees were fixed in 4% paraformaldehyde and stained for lacZ by incubating 4 hours at 37° C. in 1 mg/ml X-Gal in 2.5 mM K₄Fe(CN)₆, 2.5 mM K₃Fe(CN)₆, 50 mM Tris pH 8.0. They were then fixed in 10% formalin for 7 days. Tissues containing bone and cartilage were subsequently decalcified by incubation in EDTA. The fixed tissues were then imbedded in paraffin, sectioned at 7 μm, and stained with eosin.

### Example I

### Lentivirus-Mediated Delivery of the β-GEO Gene In Vitro and In Vivo

To determine the relative efficiency with which high-titer VSV-G pseudotyped HIV-1-based lentivirus could infect and genetically modify cells from articular tissues, a battery of cell types was infected with 5×10⁷ iu of β-GEO (β-galactosidase/neomycin resistance fusion gene) lentivirus. Primary monolayer cultures of chondrocytes and synoviocytes of human and rat origin were incubated with recombinant lentivirus at a multiplicity of infection (MOI) of 500. Forty-eight hours later, approximately 95% of cells in each culture, including human articular cells, stained positive for β-galactosidase activity. Similar levels of infection were also noted using a rabbit synovial fibroblast cell line, HIG-82, murine 3T3 cells, and primary cultures of rat skin cells. These results showed that lentivirus could indeed transduce synovial and chondrocyte cultures with reasonable efficiency in vitro and led us to evaluate its capacity for intra-articular gene transfer in vivo.

In previous studies, the rabbit knee has been typically used as an experimental model for intra-articular gene transfer (Bandara et al. (1993), supra; Ghivizzani et al. (1998), supra; Otani et al., supra; Ghivizzani et al. (1997), supra). However, given the greater availability of inbred strains, including athymic animals, and reagents, rats were used as described herein for in vivo experiments with lentivirus. For these experiments, four groups of Wistar rats were used. The first group received 5×10⁷ iu of β-GEO lentivirus by direct injection into each knee joint. The second group was injected with 5×10⁷ iu of lentivirus containing no cDNA as a negative control, and the third group was infected with 5×10⁷ plaque-forming units (pfu) of recombinant adenovirus encoding lacZ (Ad.lacZ). The latter served as a positive control for lacZ staining and provided a reference with which to compare the lentiviral vector. A fourth group of untreated naive animals was also included. Five days after injection, the rats were euthanized, and the knees processed for histological analysis.

Following intra-articular injection of 5×10⁷ iu β-GEO lentivirus in both knees, numerous superficial cells of the synovial lining of the knee joint stained positively for β-galactosidase activity (rats were sacrificed 5 days post-injection). No lacZ+ cells were observed in any other tissues of the knee joint, including cartilage. Encouragingly, the number and intensity of stained cells were similar to those achieved with the adenoviral vector (Ad.LacZ), which was injected at 5×10⁷ pfU. Synovia recovered from naive animals and those receiving 5×10⁷ iu negative control lentivirus exhibited a diffuse background staining. Relative to naive knees, no evidence of infiltration or inflammation was observed in the synovium following intra-articular injection of the lentiviral vectors.

Thus overall, the similarity in staining between the adenoviral and the lentiviral β-galactosidase vectors, and the lack of discrete cellular staining in the negative controls, showed that the lentiviral vector was capable of efficiently transducing cells in the synovium.

### Example II

### Lentivirus-Mediated Delivery of the hIL-1Ra Gene In Vitro and In Vivo

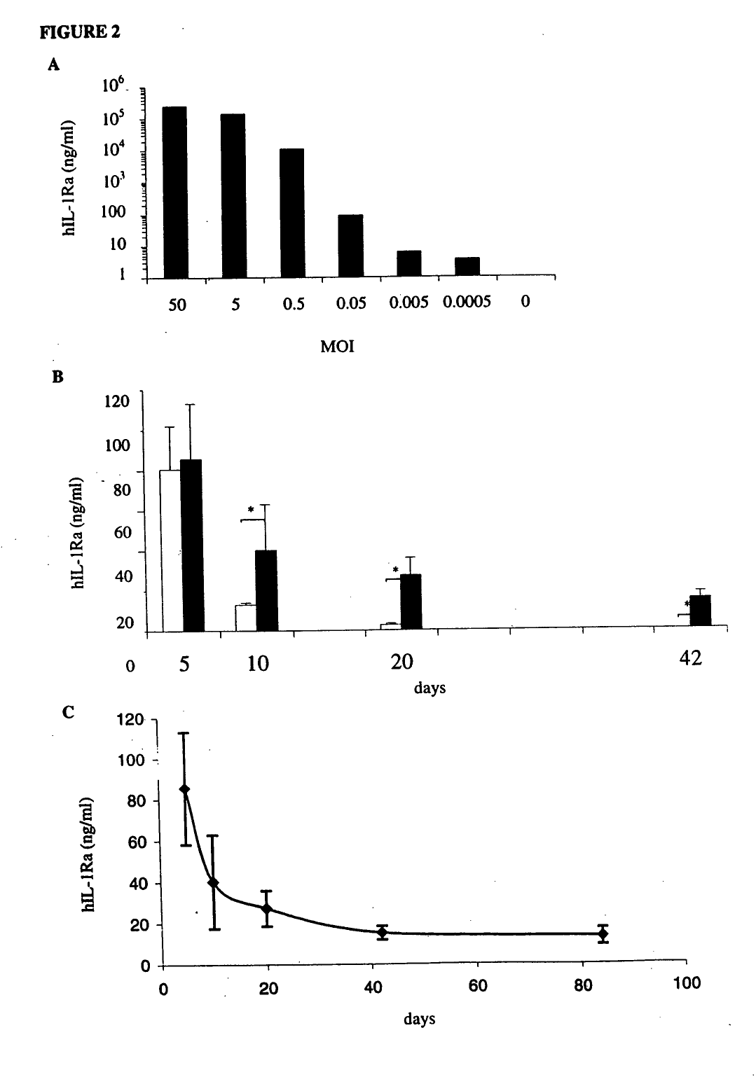

To provide a quantitative assessment of the level of intra-articular expression of a secreted therapeutic transgene afforded by lentiviral vectors, a recombinant lentivirus was constructed containing human interleukin-1 receptor antagonist (hIL-1Ra). In order to characterize the lentiviral construct containing the coding sequence of the hIL-1Ra gene, 10⁵ rat synovial cells were incubated with different amounts of recombinant lentivirus (FIG. 2A). At MOIs between 5×10⁻² and 5, the amount of hIL-1Ra produced by the synovial cells increased linearly, reaching a maximum of 2.35 μg/ml at a MOI of 50.

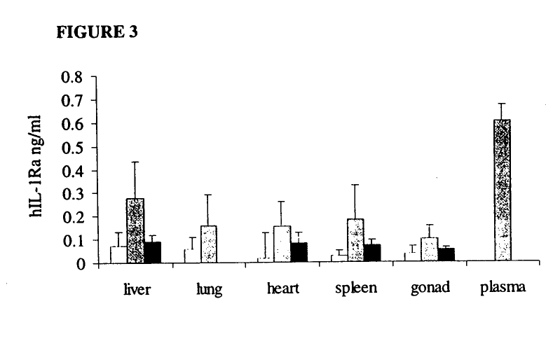

Because of the relatively small size of the rat knee joint, sufficient volumes of synovial fluid could not be recovered by joint lavage to permit measurement of secreted transgene products by ELISA. Therefore, to determine the level of intra-articular hIL-1Ra expression, knees were harvested from rats euthanized 5, 10, or 20 days following virus injection and dissected to expose the internal surfaces of the joint capsule. Dissected knee joints were washed extensively with saline and then placed into organ culture, allowing secretion of the hIL-1Ra gene product into the medium. To study the biodistribution of hIL-1Ra transgene product and of the vector, serum was collected, and the heart, liver, lung, spleen, and gonads of the animals were harvested. A portion of each tissue (approximately 100 mg) was minced with a scalpel, and placed in in 1 ml culture media for 24 hours. Blood samples were collected by cardiac puncture and centrifuged, and serum was stored at −20° C. until testing. The remainder was used for extraction of RNA. The levels of hIL-1Ra in the conditioned media and sera were then measured by commercially available ELISA that does not cross-react with the rat homolog of IL-1Ra, and compared to levels from naive animals (FIG. 3).

In addition, 5×10⁷ iu IL-1Ra lentivirus were injected into both knee joints of Wistar rats. Five days following injection of the IL-1Ra lentivirus, a mean level of 80.6 ng hIL-1Ra per ml of conditioned medium was generated by the cultured knee joints. This decreased to 12.9 ng/ml at day 10, and to 2.7 ng/ml by day 20 (FIG. 2B). Slightly elevated levels of hIL-1Ra were measured in serum, and in medium conditioned by the liver, lung, and spleen (FIG. 3). RT-PCR analyses of total RNA from these tissues were negative for hIL-1Ra message transduced from the lentiviral vector. This suggested that the levels of hIL-1Ra protein detected at day 5 in the serum, and in some organs, probably reflected escape of protein from the knees due to high levels of intra-articular transgene expression.

Because the RT-PCR assays did not provide a sensitivity beyond the detection of one transduced cell in a thousand, the biodistribution of the lentivirus was further assessed using firefly luciferase as a highly sensitive, quantitative marker gene whose product remains intracellular. A recombinant lentiviral vector encoding luciferase was injected into the knees of Wistar rats. Two days following the injection of the lentivirus, a mean level of 4.6×10⁶ RLU (relative light units) was detected in tissues recovered from the joint capsule (Table 1). This decreased to 3.3×10⁶ RLU by day 5, and 0.1×10⁶ RLU by day 10. Trace luciferase activity was also observed in several organs, however collectively the levels detected extra-articularly represented by day 2 less than 0.007% of those detected in the knees.

The biodistribution experiment described above confirmed that the transgene was expressed almost entirely within the knee joint into which it was introduced. Collectively, 1.5% of the total measured hIL-1Ra occurred in the extra-articular compartments that were analyzed; this figure fell to 0.007% for luciferase. This disparity supported the conclusion that most of the hIL-1Ra detected in the organs represented capture of circulating protein.

Because recombinant, pseudotyped lentiviruses are concentrated by ultracentrifugation of conditioned media from producer cells, there was a possibility that vector-encoded recombinant proteins expressed and secreted during viral synthesis could have been concentrated with the viral particles (Liu et al. (1996) J. Virol. 70:2497-2502). Thus, the unusually high levels of hIL-1Ra observed in media conditioned by the lentivirally injected knees could have reflected residuum from the injection of high amounts of co-concentrated protein. Indeed, ELISA measurements showed that approximately 2 μg/ml of recombinant hIL-1Ra protein was present in the viral preparation, and thus about 100 ng was injected into each knee joint along with the IL-1Ra lentiviral particles. Therefore, to test this, a series of experiments was performed to determine if the high levels of hIL-1Ra detected at day 5 were newly synthesized transgene products, or were merely the result of the release of contaminating, preformed, recombinant protein.

First, Wistar rats were injected intra-articularly with hIL-1Ra lentivirus and, following sacrifice, the harvested knees were subjected to 4 freeze-thaw cycles prior to placement in organ culture. It was rationalized that this procedure would kill the cells and that any hIL-1Ra observed in the media would arise from residual protein in the tissue and not from active synthesis. Following this treatment, approximately 1 ng of hIL-1Ra was consistently detected in the conditioned media at 5, 10, and 20 days post-injection.

To determine if it was possible for hIL-1Ra to persist in the joint following intra-articular injection, 100 ng of purified, recombinant protein was injected. No hIL-1Ra was detected following culture of the knees of the rats 5 days later, showing that the soluble protein was not retained in this environment. To investigate this issue further, we injected intra-articularly into both knee joints of Wistar rats, 5×10⁷ iu of concentrated IL-1Ra lentivirus previously inactivated by successive freeze-thaw cycles. No hIL-1Ra protein was detected in media conditioned by these knee joints 5 days after injection. Collectively, these results indicate that the high levels of hIL-1Ra we observed in the cultures of the knees injected with hIL-1Ra lentivirus were primarily due to protein synthesis by genetically modified cells and not due to the high bolus of recombinant protein co-administered with the viral vector.

### Example III

### In Vivo Expression of hIL-1Ra in Athymic Nude Rats

As described in Example II, five days post-injection into normal immuno-competent Wistar rats, high intraarticular transgene expression was observed, with transduced rat knees secreting a mean level of 80.6 ng hIL-1Ra as measured by ELISA following a 24-hrs incubation of excised knee joints in organ culture. However, as seen in FIG. 2B, between day 5 and day 10, a steep drop in lentiviral mediated hIL-1Ra production was observed in immuno-competent Wistar rats injected with lentivirus expressing human Il-1Ra.

To test whether the decrease in expression of human IL-1Ra was due to an immune response to the xenogeneic, human IL-1Ra protein (hIL-1Ra), 5×10⁷ iu IL-1Ra lentivirus was injected into the knee joints of athymic nude rats, which are T-cell deficient, as well as control immuno-competent rats. Animals were euthanized 5, 10, 20, 42 days or three months days after injection. Knees were dissected and incisions were made to allow exposure of the entire joint capsule. Joints were then placed in 24-well plates with 1 ml of DMEM and cultured for 24 hours. The hIL-1Ra content in the conditioned media was determined by ELISA. Ex vivo culture of the knees of naive animals results in mean background levels of 139.6±14.3 pg/ml. As shown in FIG. 2B, relative to day 5, hIL-1Ra production in the Wistar rats dropped by ˜85% at day 10, and by day 20, had been reduced by 95% of day 5. Expression of hIL-1Ra in the knees of the nude rats was similar to that of the Wistar rats at day 5. However, at day 10, the nude rats continued to express nearly 50% of the day 5 levels, and at day 20, 30%. Six weeks following the intra-articular injection, 15 ng/ml of hIL-1Ra were still detected in the conditioned media. Expression in athymic rats persisted for at least three months at importants levels following injection (FIG. 2C). Since lentiviral vectors do not contain coding sequences for native viral proteins, these results indicate that a T-cell mediated immune response to human IL-1Ra is at least partially responsible for the rapid decrease of expression observed in the knees of normal Wistar rats. Furthermore, this provides encouraging evidence that, in the absence of an immune reaction to a non-self transgene product, lentiviral vectors have potential for long-term expression in vivo. Importantly, these data suggest that in a homologous system, such as when a human transgene is expressed in a human joint, transgene expression can persist for a prolonged period.

Overall, while none of the existing gene delivery systems have been able to achieve long-term expression of a transgene intra-articularly, the maintenance of hIL-1Ra expression in an immuno-compromised environment demonstrates that lentiviral vectors of the current invention can achieve persistent gene expression in completely homologous systems. This indicates that in a completely homologous system where the transgene product is native to the recipient, lentiviral vectors may provide persistent expression.

### Example IV

### Effects of Lentiviral-Mediated hIL-1Ra Expression in Arthritic Rats

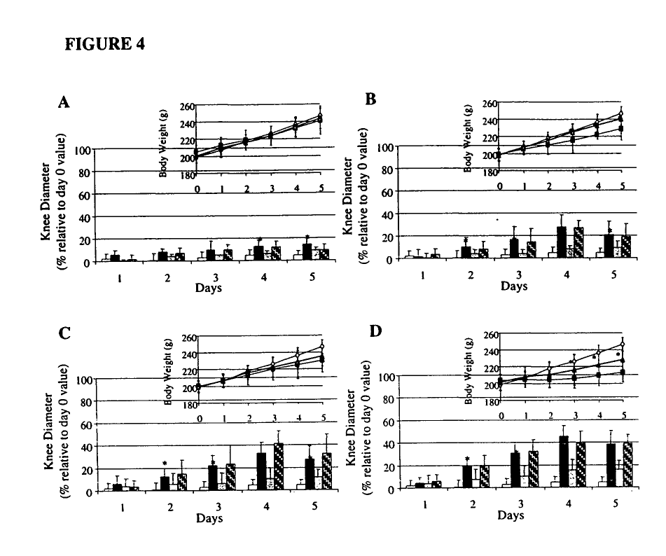

To assess the effect of lentiviral-mediated hIL-1Ra expression in arthritic rats, one knee joint of normal immuno-competent Wistar rats was injected with 5×10⁷ i.u. recombinant lentivirus containing the human IL-1Ra cDNA under the transcriptional control of the EF-1a promoter. Twenty-four hours later, arthritis was induced by bilateral intra-articular injection into both knee joints of 3×10³ (A), 1×10⁴ (B), 3×10⁴ (C) or 1×10⁵ (D) dermal fibroblasts engineered to produce hIL-1β. Knee diameters were measured daily for five days in a double blind fashion (FIG. 4). Body weights were also measured daily (FIG. 4, insets). As shown in FIG. 4, this experiment demonstrated that expression of hIL-1Ra via lentiviral injection reduces inflammation of the knee (site of injection) in arthritis induced rats compared to control animals. When 1×10⁵ dermal fibroblasts were injected into the knees of rats (FIG. 4D) with and without hIL-1Ra expressing lentivirus, there was a dramatic decrease in knee diameter in the animals injected with lentivirus.

In a further experiment, rats were injected in both the presence or absence of 5×10⁷ iu of hIL-1Ra lentivirus, with 10⁵ dermal fibroblasts engineered to produce hIL-1β to induce arthritis. Uninjected rats were also used as a control. Five days following the injection, the rats were sacrificed, and their knees were dissected. After the knee tissue was fixed in formalin and embedded in paraffin, tissues were sectioned at 7 μm, and stained with either toluidine blue to assess cartilagenous changes or hematoxylin and eosin to assess inflammation.

Knees were macroscopically observed for differences and improvements in arthritic rats injected with recombinant lentivirus. By observation, arthritic knees were characterized by severe inflammation of the synovium. Arthritic knees injected with hIL-1Ra lentivirus showed reduced swelling in comparison to knees contralateral to the lentiviral injection. The lentiviral injected knees physically resembled the naïve knees more so than the arthritic knees. Histological anaylsis using toluidine blue revealed extreme cartilage damage in the arthritic knees. This damage was not observed in the arthritic knees injected with lentiviral hIL-1Ra. Finally, sections stained with hematoxylin and eosin revealed that arthritic knees injected with lentiviral hIL-1Ra were less inflamed than uninjected arthritic knees. These results demonstrate that expression of hIL-1Ra via a lentiviral vector can prevent highly destructive experimental arthritis driven by synovial expression of IL-1.

In conclusion, the results of this study demonstrate that a VSV-G pseudotyped, HIV-1-based lentiviral vector efficiently transduces synovial lining cells following direct, intra-articular injection, that the vector achieves long-term expression of the transgene, and that expression of lentiviral delivered IL-1Ra greatly reduces the pathology observed in a rat model of rheumatoid arthritis.

Equivalents

Although the invention has been described with reference to its preferred embodiments, other embodiments can achieve the same results. Those skilled in the art will recognize or be able to ascertain using no more than routine experimentation, numerous equivalents to the specific embodiments described herein. Such equivalents are considered to be within the scope of this invention and are encompassed by the following claims.

Incorporation by Reference

The contents of all references and patents cited herein are hereby incorporated by reference in their entirety.

1. A method for treating arthritis comprising delivering to a subject a therapeutic gene using a lentiviral gene delivery vector such that the gene is expressed at sufficient levels and for a sufficient period to treat the subject.

2. The method of claim 1, wherein the lentiviral vector is selected from the group consisting of HIV, FIV, SIV, BIV, EIAV vectors.

3. The method of claim 1, wherein the therapeutic gene is selected from the group consisting of soluble Interleukin-1α Receptor Type I, Soluble Interleukin-1α Receptor Type II, Interleukin-1α Receptor Antagonist Protein (IRAP), Insulin-Like Growth Factor (IGF), Tissue Inhibitors of Matrix Metallo-Proteinases (TIP)-1, -2, -3, -4, Bone Morphogenic Protein (BMP)-2 and -7, Indian Hedgehog, Sox-9, Interleukin-4, Transforming Growth Factor (TGF)-β, Superficial Zone Protein, Cartilage Growth and Differentiation Factors (CGDF), Bcl-2, Soluble Tumor Necrosis Factor (TNF)— a Receptor, Fibronectin and/or Fibronectin Fragments, Leukemia Inhibitory Factor (LIF), LIF binding protein (LBP), Interleukin-4, Interleukin-10, Interleukin-11, Interleukin-13, Hyaluronan Synthase, soluble TNF-α receptors 55 and 75, Insulin Growth Factor (IGF)-1, activators of plasminogen, urokinase plasminogen activator (uPA), parathyroid hormone-related protein (PTHrP), and platelet derived growth factor (PDGF)-AA -AB or -BB

4. The method of claim 1, wherein the lentiviral vector is injected directly into an affected joint of the subject.

5. A method for treating arthritis comprising transfecting cells ex vivo with a therapeutic gene using a lentiviral gene delivery vector and administering the cells to a subject.

6. The method of claim 5, wherein the lentiviral vector is selected from the group consisting of HIV, FIV, SIV, BIV, and EIAV vectors.

7. The method of claim 5, wherein the therapeutic gene is selected from the group consisting of soluble Interleukin-1α Receptor Type I, Soluble Interleukin-1α Receptor Type II, Interleukin-1α Receptor Antagonist Protein (RAP), Insulin-Like Growth Factor (IGF), Tissue Inhibitors of Matrix Metallo-Proteinases (TIMP)-1, -2, -3, -4, Bone Morphogenic Protein (BMP)-2 and -7, Indian Hedgehog, Sox-9, Interleukin-4, Transforming Growth Factor (TGF)-β, Superficial Zone Protein, Cartilage Growth and Differentiation Factors (CGDF), Bcl-2, Soluble Tumor Necrosis Factor (TNF)-α Receptor, Fibronectin and/or Fibronectin Fragments, Leukemia Inhibitory Factor (LIF), LIF binding protein (LBP), Interleukin-4, Interleukin-10, Interleukin-11, Interleukin-13, Hyaluronan Synthase, soluble TNF-α receptors 55 and 75, Insulin Growth Factor (IGF)-1, activators of plasminogen, urokinase plasminogen activator (uPA), parathyroid hormone-related protein (PTHrP), and platelet derived growth factor (PDGF)-AA -AB or -BB

8. The method of claim 5, wherein the cells are autologous.

9. The method of claim 8, wherein the cells are bone marrow cells.

10. The method of claim 8, wherein the cells are mesenchymal stem cells obtained from adipose tissue.

11. The method of claim 8, wherein the cells are synovial fibroblasts or chondrocytes

12. The method of claim 5, wherein the cells are non-autologous (allogeneic or xenogenic).

13. The method of claim 12, wherein the cells are a cell line or primary cells derived from a human or animal source.
